(19) United States
(12) Patent Application Publication
(10) Pub. No.: US 2023/0025309 A1
(43) Pub. Date: Jan. 26, 2023

(54) METHOD FOR MANUFACTURING A PHOTOCATALYTIC DEVICE, PHOTOCATALYTIC DEVICE, PHOTOCATALYTIC COMPOSITION AND GAS DEPOLLUTING APPARATUS

(71) Applicant: CALISTAIR SAS, Vaux-le-Pénil (FR)

(72) Inventor: Jérôme TARANTO, Dormelles (FR)

(21) Appl. No.: 17/787,800

(22) PCT Filed: Dec. 28, 2020
(86) PCT No.: PCT/EP2020/087920

§ 371 Date: Jun. 21, 2022

(30) Foreign Application Priority Data
Dec. 27, 2019 (FR) FR1915667
Jul. 24, 2020 (EP) PCT/EP2020/070931

(51) Int. Cl. B01J 35/00; B01J 21/06; B01J 29/70; B01J 23/34; B01D 53/88; B01J 37/00; B01J 37/04

(52) U.S. Cl. CPC B01J 35/004; B01J 21/063; B01J 29/7003; B01J 23/34; B01D 53/885; B01J 37/0045; B01J 37/04; B01D 2253/108; B01D 2255/20707; B01D 2255/2073; B01D 2255/802; B01D 2258/06; B01D 2259/804

(57) ABSTRACT

The invention refers to a method for manufacturing a catalytic device, with the steps: a) providing a first catalyst having photocatalytic activity, a second catalyst, which is a different molecule than the first catalyst, and an adsorbent, each in a powdered state, b) mingling the first catalyst, the second catalyst and the adsorbent to form a catalytic composition and suspending them in a suspension liquid to form a slurry, and c) repeatedly coating the slurry onto a solid grid-like carrier having a plurality of through holes, configured to allow a gas to flow through the carrier, and evaporating the suspension liquid.

The invention relates to a method for manufacturing a catalytic device. According to another aspect the invention relates to a catalytic device obtainable via such a method. According to another aspect the invention relates to a catalytic composition. According to another aspect the invention relates to a gas depolluting apparatus, comprising such a catalytic device and/or catalytic composition.

### PRIOR ART

The depollution of gases is widely used and needed. Contaminants within gases, especially within air, can be of various types. They can be divided into particles, gaseous or volatile chemical contaminants and biological contaminants. Biological contaminants are microorganisms such as bacteria, fungi including their spores or mites as well as viruses, phages and the like.

Gaseous or volatile substances are for example the so called Volatile Organic Compounds (VOC). VOC are usually defined as compounds that contain at least one carbon atom and one or more atoms of hydrogen, halogen, oxygen, sulfur, phosphorus and/or silicon and that have a vapor pressure of at least 0.01 kPa at a temperature of 20° C. or a certain volatility under certain circumstances. Carbon oxides and inorganic carbonates or bicarbonates are usually not considered to be VOC. VOC therefore represent a wide range of different substances which have in common that they easily evaporate or sublime. Examples of volatile or gaseous contaminants are formaldehyde, carbon monoxide, nitrogen oxides, ozone or radioactive substances like radon.

One way to treat and depollute gas, especially air, is the use of mechanical filters such as HEPA filters or the like. These are filtering particulate contaminants as well as some biological contaminants. Therefore they have the disadvantage that they need to be cleaned or replaced after a certain time. Moreover, these filters are less efficient towards particles having a size between around 0.1 and 1 micron, that is the size of most viruses. Furthermore, microbiological contamination of the filters can lead to the production and secretion of possibly harmful or toxic substances from bacteria or the like, which are then carried with the gas flow. For example, these toxic substances can be endotoxins formed after lysis of some gram-negative bacteria cell walls or VOC from microorganism decomposition on filters. Vulnerable microorganisms such as Staphylococcus epidermidis, E. coli or Brevundimonas diminuta can survive for 2-6 days on such a filter. Robust biological contaminants such as Bacillus atrophaeus, MS-2 Coliphage, and Aspergillus brasilensis survive more than six days. Bacillus atrophaeus can remain viable for more than 210 days. Therefore the use of such filters can lead to an additional contamination of the gas to be cleaned. Also the biological contaminants can lead to a saturation of the filter which leads to an increased flow resistance. Moreover, the saturation of the filter by biological contaminants, which are filtered and afterwards additionally colonizing the filter surface, can lead to a global decrease of the performance of a gas depolluting apparatus. Problems that can occur are odors, a decrease of the air flow, an increase of the pressure drop and the formation of biofilms on internal surfaces of the filter and the gas depolluting apparatus in general. Nevertheless such filters can be useful to eliminate particulate contaminants.

Several further solutions for air depollution or purification are proposed, e.g. the use of plasma, ionization, ozonization or catalysis.

In ionization technology a high energy is used to form oxygen ions, e.g. by a high voltage or with specific lamps. These accumulate on the surface of particulate contaminants, such as dust, and promote the formation of larger clusters, which can then be filtered or descend to the floor.

Therefore the contaminants are not eliminated or cracked but may remain in clusters for example on a floor and can be later still airborne. Furthermore ozone can be generated which on the one hand is disinfecting but on the other hand may cause allergies or be harmful for people nearby.

During ozonation ozone is formed which is highly reactive and a strong oxidizing agent. It reacts both with microbiological and chemical contaminants. A disadvantage is that not all of the generated ozone directly reacts with contaminants so that again ozone may be harmful to people nearby.

Catalysts are used in many industrial fields. A catalyst is a substance which is able to catalyze reactions, namely to increase the rate of a chemical reaction without being consumed in the reaction. One specific field is the use of catalysts for depolluting gases such as air, especially ambient air, or exhaust or process gases. They can be used in air conditioning systems or air purifying systems for cleaning ambient air in hospitals, quarantine stations or public places like restaurants or public authorities and the like.

It is possible to use a single catalyst or to combine more than one catalyst, depending on the reactions that are to be catalyzed.

Catalysts having a photocatalytic activity are known in the state of the art. They are often referred to as photocatalysts and are usually semiconductors. Usually a photocatalyst is UV activatable. When the UV activatable photocatalyst is irradiated with UV radiation, namely electromagnetic radiation with a wavelength of about 100 nm to about 400 nm, it is then activated and therefore able to catalyze reactions. On the surface of the catalyst, for example water is reacted to highly reactive OH-radicals, which are then reacting with contaminants. Furthermore molecular oxygen can be reacted to the so called ROS (reactive oxygen species), especially the superoxide anion. The reactive species are reacting both with chemical and biological contaminants and for example destroy the lipid bilayer of the membranes of viruses.

When using photocatalysts for gas depollution, usually a source of UV radiation needs to be present. These radiation sources usually consume a high amount of energy and only have an energy efficiency of 30-40%. Thus, only 30-40% of the energy is used to activate the photocatalyst.

It is for example known in the state of the art, to provide separate stages of catalysts, for example a combination of three distinct stages being spatially apart from each other. Each stage contains a specific compound, for example one stage contains titanium dioxide, one stage contains manganese monoxide and one stage contains a zeolite. These are provided one after another in a direction of flow of a gas to be depolluted.

For example from CN 106582265 A and CN 105597528 A catalytic compositions for gas depollution are known. These consist of an adsorbent which is loaded with precursors of manganese oxide, e.g. a manganese acetate, and partly with a precursor for titanium dioxide, e.g. butyl titanate. Afterwards the adsorbent with the precursors is calcined at temperatures of 300° C. or higher, thereby forming manganese oxide and titanium dioxide. Due to the high calcination temperature, predominantly or solely manganese dioxide is formed.

From JPH11-137656 for example a catalytic composition is known, consisting of a manganese oxide, titanium dioxide and an adsorbent. These are bound to a solid support via a binder. The binder partly covers the catalysts and therefore only a reduced amount can be reached by activating UV radiation and the molecules to be cracked. According to another embodiment, a suspension of the components is impregnated into a porous body or a fabric. These however provide a high flow resistance to a gas to be depolluted. Therefore it is not possible to efficiently clean large amounts of gases per time unit. In the implementation of this document, the ratio second catalyst may not exceed an amount of 22% of the titanium dioxide in order not to deteriorate the activity of the titanium dioxide.

It this therefore an objective of the invention to provide a catalytic device that is easy to manufacture and that has a high efficiency in depolluting a gas.

The invention achieves this objective with a method for manufacturing a catalytic device, with the steps: a) providing a first catalyst having photocatalytic activity, a second catalyst, which is a different molecule than the first catalyst, and an adsorbent, each in a powdered state, b) mingling the first catalyst, the second catalyst and the adsorbent to obtain a catalytic composition and suspending them in a suspension liquid to form a slurry, and c) repeatedly coating the slurry onto a solid grid-like carrier having a plurality of through holes, configured to allow a gas to flow through the carrier, and evaporating the suspension liquid.

The first catalyst and the second catalyst are different molecules. This means, that the first catalyst and the second catalyst are different in their chemical composition. First and second catalyst cannot be identical compounds having different properties, like different particle size, crystal structures, shapes or the like. In other words, a composition comprising one compound in different shapes, crystal structures or the like cannot be considered as comprising two different catalysts as claimed herein.

A catalyst having photocatalytic activity is a substance that is factually capable of being photo-activated by certain irradiation as, for example, specified below. In other words, a substance having photocatalytic properties but which is not capable of being photo-activated, e.g. because it is embedded in a matrix or is coated, and therefore is factually not capable of being photo-activated is not considered a catalyst having photocatalytic activity. Titanium dioxide, for example, is used as a scattering agent in cosmetic industry. This is usually coated, in order to suppress the photocatalytic activity. Photocalatyic activity of these particles would lead to the generation of ROS, which is undesired and harmful in cosmetics. Such coated titanium dioxide particles are therefore not considered catalysts having photocatalytic activity, since they factually cannot be photo-activated. This also accounts for such particles being embedded in a matrix, which does not allow radiation to reach the particles and, thus, to photo-activate them. Nonetheless a substance which is factually capable of being photo-activated, is considered a catalyst having photocatalytic activity, even if it is not irradiated and therefore activated.

The applicants have surprisingly discovered that a mixture of the first catalyst, the second catalyst and an adsorbent each in a powdered state can—if suspended in a suspension liquid, which for example is demineralized water or a solution of demineralized water and ethanol, and then repeatedly applied to a solid carrier, adheres well to the carrier and forms an effective catalytic device.

An advantage of the combination is that it has a high depolluting efficiency and at the same no microbial growth takes place on the catalytic device as is the case on simple filters. Furthermore it is possible to not only destroy contaminants but also byproducts of the destruction process which can be undesired or harmful as well. Preferably it is possible to depollute gases that contain only a few ppb up to a few hundred ppm of contaminants.

The first catalyst, the second catalyst and the adsorbent are provided as distinct powders. On the one hand this makes it easier to handle, since no precursors or the like are to be impregnated onto another substance. On the other hand in some embodiments this is chemically necessary, since for example calcination temperatures of the individual components are different so that synthesizing two different components at the same time can be impossible.

The mingling of the first catalyst, the second catalyst and the adsorbent is preferably performed before suspending them in the suspension liquid. Thus, first a mixture of the components is made, which is then introduced into the suspension liquid to form the slurry. Alternatively it is also possible to introduce some or all components in a non-mingled state into the suspension liquid and perform the mingling of all components within the suspension, for example using a blender.

The suspension liquid is preferably a liquid or solution in which none of the components is soluble. This is preferably the case when the solubility of the first catalyst, the second catalyst and the adsorbent each lies below 1 g/l of the suspension liquid, more preferably below 0.1 g/l.

The slurry is repeatedly applied to the grid-like carrier. This means that it is applied in more than one layer and applied at least twice, preferably at least five times, more preferably at least ten times. Preferably the slurry is not applied more than 100 times, more preferably not more than 50 times. That the slurry is applied repeatedly means preferably that in each repetition or passage at least 50% of the surface, more preferably at least 75% of the surface, most preferably at least 90% of the surface, especially each time the complete surface is coated with slurry.

Each time the slurry is coated onto the grid-like carrier it forms a layer on the surface of the carrier and/or the layer of coating below. By applying the coating repeatedly it is possible to achieve a coating that provides enough catalyst and adsorbent for an efficient depollution. Furthermore the coating becomes preferably not too dense so that many of the catalyst molecules are available for reactions and for activation with UV radiation. Preferably at least 95%, more preferably at least 99% of the surface of the grid-like carrier is coated. This especially accounts for the inner surfaces of the through holes within the carrier. In use for depolluting a gas, the gas is guided through the through holes and thereby the contaminants come in contact with the catalysts and the adsorbent coated onto the inner surfaces.

Preferably, after each application of the coating, the suspension liquid is evaporated, for example by heating or preferably under air flow especially without additional heating. This obviously includes capillary bound suspension liquid, which is not evaporated during a normal evaporation process, e.g. at 100° C.-150° C. In another embodiment, the suspension liquid is not fully but partly evaporated, for example at least 75% of the suspension liquid is evaporated, before a next layer is applied. In another embodiment no suspension liquid or only a part of the suspension liquid not exceeding 25% is evaporated before applying a new layer. The less suspension liquid is evaporated before applying a new layer, the less time consuming is the procedure. The best results are usually obtained after evaporating as much suspension liquid as possible before applying a new layer.

The carrier can for example be formed from a metal, such as aluminum or steel, a plastic material or a composite material. It can for example be formed as a grid, a plate or an expanded metal. In a preferred embodiment, the carrier is shaped from a corrugated and/or folded sheet forming a plurality of through holes, preferably in a honeycomb manner. Advantages of a honeycomb panel compared to a plane material are a larger surface to be coated and a lower pressure drop. Also the irradiance within the cells is better. The carrier is then often simply referred to as a honeycomb or honeycomb panel. Preferably the through holes have a hexagonal cross section.

In a preferred embodiment the through holes account for at least 80% of the volume of the carrier, more preferably for at least 85%, most preferably for at least 90%, especially of at least 95% of the volume of the carrier. This helps to ensure a sufficient gas flow through the carrier in use. Therefore the supporting structure of the carrier is provided thinly, for example in the form of thin metal, preferably aluminum, or another inert material. Aluminum provides the advantage of being lightweight and sufficiently inert, so that the catalysts are not attacking the carrier itself in a critical manner.

The through holes preferably have a length, along which the gas can flow through the carrier, of at least 1 cm, more preferably at least 2 cm. Preferably the length does not exceed 20 cm, more preferably 10 cm. The length of the holes, which usually equals the thickness of the grid-like carrier, has great influence on the efficiency of the depollution. If the length is too short, the catalytically active surface provided is low and therefore the depollution is not efficient. If the length is too high, the UV radiation will not or not sufficiently reach the photocatalyst in the center of the holes, so that a part of the provided photocatalyst is not activated. Furthermore it is difficult to satisfactorily coat the center parts of long through holes. Therefore also the ratio between length and diameter of the holes can be important. The ratio is preferably larger than 2, more preferably larger than 5, especially larger than 10 and preferably not larger than 50, more preferably not larger than 30, especially not larger than 20. If the cross section of a through hole is not circular, the diameter of an approximated circumscribed circle or a circle approximated via the least squares method may be used to determine the diameter for calculation of the ratio. The diameter of the holes preferably is at least 2 mm, more preferably at least 5 mm, especially at least 7 mm. Preferably the diameter of the holes does not exceed 50 mm, more preferably 20 mm, especially 10 mm.

The through holes preferably run straight and are not curved or angled along their length.

The ratio between the diameter of the through holes and the thickness of the bars or sheets of the carrier separating neighboring through holes is preferably larger than 10, more preferably larger than 20, especially larger than 40.

In a preferred embodiment, the slurry is binder-free. This means that no additional inorganic or organic binder is added, to enhance the adherence of the first catalyst, the second catalyst and the adsorbent on the carrier. Binders for example are cellulose and its derivatives, certain proteins or polymers and inorganic binders such as silica or alumina. Residual amounts of the suspension liquid, for example capillary bound, are not considered a binder. Also water, for example deriving from atmospheric humidity, is not considered a binder. Preferably a slurry is still considered binder-free, as long as it does not exceed an amount of binder of 2% in relation to the total mass of the adsorbent, the first catalyst and the second catalyst. Especially there is no matrix of binder molecules formed around the catalysts and the adsorbent on the carrier after evaporating the suspension liquid.

The slurry can be applied to the carrier by various methods, including dipping. In a preferred embodiment the slurry is applied via spray coating. Spray coating resulted in a very durable and consisting coating, especially without the need for providing a separate binder to the slurry. The coating preferably resists an air pressure of up to 4 bars, especially without using a binder. The slurry is preferably applied with a spray gun. Preferably a manual spray gun is used, which especially is a low-medium pressure spray gun, for example with a maximum pressure Pₘₐₓ of 8 bar. It preferably has a gravitational suction cup. The spray coating is preferably performed vertically. In one preferred embodiment, the inlet pressure is 3.5 bar and the liquid flow is 2.5-3.0 liters per minute. The distance between the carrier and the spray gun preferably lies between 10 and 30 cm, preferably at about 15 cm. With the above mentioned exemplary parameters, good coating properties were achieved. Specifically the coating adhered satisfactorily to the carrier, which preferably is an aluminum honeycomb.

The coating preferably has a total thickness of at least 50 μm, preferably at least 75 μm, especially at least 100 μm. A coating of such thickness especially provides a satisfactory amount of catalyst for gas depollution. Preferably the thickness does not exceed 500 μm, more preferably does not exceed 350 μm, and especially does not exceed 250 μm. A preferred range lies between 100 μm and 250 μm. If the coating is too thick, it is possible that lower layers are not sufficiently irradiated and/or not sufficiently reached by contaminants to be depolluted. It can therefore lead to a waste of catalytic molecules.

The first catalyst can for example consist of tungsten oxide and/or zinc oxide. It is preferred but not necessarily the case that only one type of first catalyst is used. It is also possible to use more than one catalyst having photocatalytic activity.

In a preferred embodiment the first catalyst is titanium dioxide TiO₂. Titanium dioxide is very well known for its photocatalytic properties. It is a semiconductor and can be activated by irradiation with UV radiation of a wavelength from 100 nm to 400 nm. Preferably the UV radiation used for activating the first catalyst, especially titanium dioxide, has a wavelength of 365 nm or less, more preferable in the UV-C range between 100 nm and 280 nm, most preferably of 254 nm. The use of UV radiation in the UV-C range has the advantage of additionally using the direct disinfecting properties of the UV-C radiation. UV-C radiation therefore acts directly biocidal due to its high energy and indirectly biocidal as well as on non-biological contaminants by activating the first catalyst. The titanium dioxide can be already photo-activated.

For the activation of the first catalyst, preferably any kind of UV radiation emitting sources or light sources can be used, for example artificial lamps emitting UV radiation or light emitting diodes or fluorescent tubes emitting UV radiation or UV radiation formed by cold plasma-type electrodes.

Titanium dioxide has the advantage of being cost-efficient and having the ability to at least partly regenerate itself. It is highly active against different contaminants, including chemical and biological contaminants.

It is known that titanium dioxide in the crystalline form of anatase has the highest catalytic activity. Therefore it is one embodiment of the invention that the titanium dioxide completely is of the anatase type.

Explorations of the applicant on the other hand have shown that a certain proportion of titanium dioxide of the rutile type, against the prevailing opinion, is enhancing the overall depolluting capacities of the catalytic device. The separation of the electric charges on the surface of the catalyst is augmented and so is its efficiency.

It is a preferred embodiment of the invention that the first catalyst is titanium dioxide TiO₂ in the form of a mixture of anatase and rutile with an anatase/rutile ratio between 60/40 and 99/1. Preferably the ratio is at least 60/40, more preferably at least 70/30 and especially at least 80/20. The ratio is preferably not exceeding 99/1, more preferably not exceeding 95/5, and especially not exceeding 90/10. In a further preferred embodiment the ratio is between 77/23 and 83/17, especially 80/20.

In a preferred embodiment the first catalyst, especially titanium dioxide, is doped, especially with silver ions or platinum ions. Doping of the first catalyst increases the biocidal effect on biological contaminants and on the destruction of possibly harmful byproducts of the destroyed contaminants. The presence of the doping ions increases the number of possible oxidation and reduction reactions.

The titanium dioxide preferably has an elementary particle size of 10-50 nm, more preferably of 15-35 nm, especially around 25 nm. These elementary particles tend to aggregate. The average particle size of these aggregates preferably ranges between 200 and 600 nm, more preferably, between 300 and 500 nm, especially around 420 nm. This, however, does not exclude that some aggregates have a particle size of 1 micron or more.

In a preferred embodiment the second catalyst is a low-temperature catalyst. Low-temperature catalysts are activated by calorific energy. The term low-temperature catalyst is thereby used to differentiate this type of catalysts, which are activated at relatively low temperatures, from thermal catalysts, which are activated at relatively high temperatures of usually between 500° C. and 1200° C. A low-temperature catalyst according to the invention therefore is not only catalytically active at a relatively low-temperature but it is activated by the calorific energy at this relatively low temperature. In other words, a photocatalyst which is capable of being photo-activated at ambient temperature is no low-temperature catalyst, because it is not activated by the calorific energy at ambient temperature but by the irradiation. A low temperature catalyst preferably is a catalyst being already catalytically activated and, thus, active, at temperature lower than 100° C., more preferably at temperatures lower than 50° C., most preferably already at room temperature of 20° C. This does however not imply, that it must be inactive at higher temperatures. Preferably the catalytic activity is increased with increasing temperature at least over a certain temperature interval, preferably 20° C. to 100° C. or 50° C. to 100° C.

Low-temperature catalysts are for example metal oxides like nickel oxide or cerium oxide. In a preferred embodiment of the invention the second catalyst is manganese monoxide MnO, which is an efficient low-temperature catalyst. Explorations of the applicants have shown that manganese monoxide is significantly more efficient than the well-known manganese dioxide MnO₂, whose catalytic activity is insufficient. Therefore preferably the manganese monoxide used in this embodiment is not or substantially not contaminated with manganese dioxide. In a preferred embodiment, the amount of manganese dioxide is lower than 5%, more preferably lower than 1%, most preferably lower than 0.1% of the total mass of the manganese monoxide used as second catalyst. Optimally, no manganese dioxide is present as an impurity of the manganese monoxide second catalyst.

Manganese monoxide, especially in crystalline form, allows the generation of highly reactive radical species when in contact with oxygen, for example from the air. Preferably the temperature is higher than 35° C., especially between 35° C. and 55° C., more preferably between 45 and 50° C. Preferably the relative humidity of the gas to be depolluted, especially air, is between 30-80%, more preferably 50%. Under these circumstances a very efficient generation of the radical species is possible. The radical species are able to react also with very small contaminants in the nano or micro scale, such as aldehydes like formaldehyde, which are usually hard to crack. This is, inter alia, because such small molecules are hard to be trapped. Hydrophilic catalysts are usually quickly saturated with water or other polar small molecules so that hardly any sites are available for small contaminants to be trapped and then oxidized. Manganese monoxide offers the specific advantage that it is only poorly reacting with water and trapping water molecules on its surface so that more sites are available for contaminants. Furthermore, the cavities within the manganese monoxide are preferably smaller than those of the titanium dioxide so that larger molecules are trapped less and, again, more sites remain available for small contaminants. The radical species also react with biological contaminants.

A disadvantage of manganese monoxide is that it is unstable compared to manganese dioxide. Therefore, when synthesizing manganese monoxide, special care needs to be taken on the reaction parameters, when the formation of manganese dioxide is to be avoided, as preferred. One important reaction parameter is the temperature. When using a calcination temperature higher than 300° C., a substantial amount or solely manganese dioxide is formed.

Since the formation of titanium dioxide from precursors usually requires temperatures of more than 300° C., for example 300° C.-600° C., a simultaneous calcination of manganese monoxide precursors and titanium dioxide precursors to form manganese monoxide on the one hand and titanium dioxide on the other hand, is impossible. A calcination temperature of higher than 600° C. during the formation of the titanium dioxide may lead to an excessive formation of rutile, which is undesired. On the other hand, a certain amount of rutile, as described above, enhances the efficiency of the catalyst.

Therefore, when the first catalyst is titanium dioxide and the second catalyst is manganese monoxide, preferably no simultaneous calcination takes place.

It is preferred but not necessarily the case, that the synthesis of the first catalyst, the second catalyst and/or the adsorbent is part of the manufacturing method of the catalytic device. Preferred embodiments of the synthesis will be explained in detail below. If at least the synthesis of titanium dioxide as first catalyst and manganese monoxide as second catalyst is part of the manufacturing method, no simultaneous calcination of their precursors preferably takes place. They are preferably synthesized apart from each other.

One advantage of using a low-temperature catalyst, especially manganese monoxide, is that the necessarily accruing waste heat of the UV radiation source can be used to warm the low-temperature catalyst thereby increasing its catalytic efficiency. Hence, a synergy can be achieved when combining a photocatalyst and a low-temperature catalyst in one stage and preferably arrange them spatially close to the UV radiation source in order to use the waste heat efficiently. Preferably a temperature of up to 40° C., up to 50° C. or up to 90° C. can be achieved on the surface of the catalytic device.

If, as preferred, the second catalyst is manganese monoxide, its average particle size preferably is between 50 and 170 nm, more preferably between 95 and 135, especially around 110 nm.

The adsorbent is preferably a compound having a large specific surface area, preferably of at least 300 m²/g, more preferably of at least 500 m²/g, most preferably of at least 1000 m²/g, especially more than 2000 m²/g. The adsorbent can for example be activated carbon or activated coke.

In a preferred embodiment the adsorbent is a zeolite. Zeolites are microporous aluminosilicate minerals which can be naturally occurring or artificial. Zeolites can be synthetic.

Preferably a hydrophilic zeolite is used, as explorations of the applicant show that biological contaminants tend to be better adsorbed by a hydrophilic than a hydrophobic zeolite. Preferably a zeolite of type A or ZSM-5 is used. The zeolite is preferably a synthetic zeolite, especially a synthetic zeolite of type A or ZSM-5. Synthetic zeolite has the advantage of being pure and having a homogenous structure due to the fact that is synthesized.

In a preferred embodiment of the invention, the first catalyst is titanium dioxide, the second catalyst is manganese monoxide and/or the adsorbent is a zeolite. The zeolite is preferably a synthetic hydrophilic zeolite of type A. These configurations are preferred for all applicable embodiments described herein.

The applicants have found, that certain ratios of the single components are advantageous, for efficient elimination of contaminants. Preferably the ratio between the zeolite of type A and titanium dioxide ranges between 3:1 and 1:1, especially is around 2:1. The ratio of the zeolite of type A and manganese monoxide preferably ranges between 5:1 and 3:1, especially is around 4:1. The ratio of titanium dioxide and manganese oxide preferably ranges between §:1 and 1:1, especially is around 2:1. These preferred ratios also apply for first catalysts, second catalysts and adsorbents other than titanium dioxide, manganese monoxide and/or the zeolite of type A.

Hydrophilic zeolite of type A has a high affinity to chemical and biological contaminants and no natural equivalent. It has an affinity to the cellular membranes of microorganisms. These adsorb to the surface of the zeolite for electrostatic reasons. Additionally, the hydrophilic zeolite of type A has a direct antimicrobial effect, which makes its use even more synergistic. Hydrophilic zeolite of type A has a crystal structure formed from an anionic aluminosilicate structure, neutralized by alkaline or alkaline earth metal cations.

Preferably the zeolite, especially the zeolite of type A comprises a sodalite crystal structure.

The structure of the zeolite links multiple elementary sodalite cages. A sodalite cage consists of multiple polyhedrons with eight hexagonal faces and six square-shaped faces. The sodalite cages forming the zeolite are interconnected via the square-shaped faces. The specific structure of the sodalite cages gives the zeolite an open 3D structure, whereby especially 47% of the total volume is formed by interstices. Therefore the zeolite provides a large surface for adsorption of biological and chemical contaminants within a small volume. Additionally the open 3D structure allows for a high water absorption and retention capacity. Since water can be used to form highly reactive radical species like the hydroxyl radical on the surface of the catalysts, this can be advantageous. Furthermore the water retention capacity may, depending on the circumstances, ensure that no water film is formed on the catalysts, thereby degrading their efficiency. On the other hand a very high amount of adsorbent, especially zeolite, may provide for a water excess, thereby degrading the efficiency of the catalysts. Preferably the amount of adsorbent therefore does not exceed 80%, especially does not exceed 70% of the total mass of first catalyst, second catalyst and adsorbent. On the other hand, if the amount of adsorbent is very low, the water retention capacity might not be sufficient. Therefore the amount of adsorbent preferably is not lower than 40% especially not lower than 30% of the total mass of first catalyst, second catalyst and adsorbent.

The applicants have explored that the combination of two different catalysts and an adsorbent is synergistically effective for depolluting a gas. The contaminants are adsorbed onto the adsorbent. By way of the mass transfer phenomenon, they migrate to the catalyst particles. The generation of reactive species on the surface of the catalysts then leads to destruction of the contaminants, preferably to complete mineralization of the contaminant. Also byproducts during the destruction of the contaminants are preferably destroyed further so that no possibly harmful byproducts are released into the depolluted gas or are released but destroyed within one of the next depolluting cycles. The adsorbent can thereby also form a reservoir for pollutants to be destroyed, for example during a peak load of the gas with contaminants. An amount of contaminants that would exceed the catalytic capacity of the catalysts per time unit can therefore preferably be held back by the adsorbent and then be guided to the catalysts with a time delay. This makes it possible to also treat peak loads that would, without adsorbent, exceed the capacity of the catalysts per se. In order to provide good adsorbing properties, the amount of the adsorbent is preferably higher than the amount of each catalyst and/or the cumulated amount of the catalysts, with respect of the total mass of the first catalyst, the second catalyst and the adsorbent.

The adsorbent is holding back contaminants which are then transferred to the catalysts via mass transfer. On the other side, by this phenomenon, the adsorbent is constantly regenerated and its adsorbing capacity restored. This demonstrates a real synergy of providing these compounds within one mixture. In order to optimally achieve this effect, the mingling of the catalysts and the adsorbent is preferably performed intensively, especially to provide a mixture as homogenous as possible.

The amount of the first catalyst is preferably at least 10%, more preferably at least 20% and does preferably not exceed 30%, especially does not exceed 40% of the total mass of the first catalyst, the second catalyst and the adsorbent. The amount of the second catalyst is preferably at least 5%, more preferably at least 10%, especially at least 15% and does preferably not exceed 20%, especially does not exceed 30% of the total mass of the first catalyst, the second catalyst and the adsorbent.

In a preferred embodiment, the components are provided in the following ranges, in weight percent with regard to their total mass: Between 27% and 30% of the first catalyst, between 11% and 17% of the second catalyst and between 55% and 59% of the adsorbent.

A specific and preferred embodiment contains an amount of 29% of the first catalyst, 12% of the second catalyst and 59% of the adsorbent.

The explorations of the applicants showed that this ratio shows a good efficiency as well as satisfying water retention capacities and contaminant retention capacities. With these amounts an especially sustainable and long lasting composition is provided that also provides very good depolluting attributes. Additionally, the amount of UV radiation needed to activate the titanium dioxide in this amount provides for a sufficient and useful amount of waste heat to increase the catalytic activity of the second catalyst in this amount, when the second catalyst is a low-temperature catalyst.

Additionally, this invention relates to an embodiment in which the amount of the first catalyst is lower than 27% and higher than 30%, the amount of the second catalyst is lower than 11% and higher than 17% and the amount of the adsorbent is lower than 55% and higher than 59%, each in weight percent and with regard to their total mass. All further preferred configurations and embodiments described herein apply to this specific embodiment.

As stated above it is preferred but not necessary, that the synthesis of the first catalyst, the second catalyst and/or the adsorbent is part of the manufacturing method of the catalytic device. The syntheses each are performed apart from another.

If the first catalyst, as preferred, is titanium dioxide, it is preferably synthesized by using a sol-gel process and precursors like titanium tetrachloride or butyl titanate, followed by a calcination at temperatures of 300-600° C. Also different methods for producing powdered titanium dioxide like thermal plasma technology, laser pyrolysis, hydrothermal or electrochemical synthesis are possible.

A preferred sol-gel process for synthesizing the titanium dioxide first catalyst in a powdered form is now described in detail:

The precursor titanium tetrachloride (obtained from Merck, ref. 697079) is mixed with absolute ethanol obtained from Merck, ref. 1024282500) in a ratio between 0.5:10 and 3:10, preferably with a ratio of 1:10, for 4 h at room temperature. The formed sol is then placed in an ultrasonic bath for a time of 20 to 40 minutes, preferably 30 minutes. Consecutively the sol is dried at a temperature of 100° C. and 130° C., preferably 120° C. for a time between 1 h and 24 h, preferably 7 h in order to obtain a powder. This powder is then progressively calcined at a temperature between 530 and 570° C., preferably at 550° C., for 2 h to 3 h, preferably 2 h. Thereby the temperature is increased in steps of 10-12° C./minute. The obtained powdered titanium oxide is then cleansed with deionized water and consecutively dried at a temperature between 90 and 110° C., preferably 100° C.

If the second catalyst is, as preferred, manganese monoxide, it is preferably synthesized from precursors like manganese acetate, manganese nitrate and/or manganese sulfate followed by a calcination at temperatures below 300° C.

A preferred process for synthesizing the manganese monoxide second catalyst in a powdered form is now described in detail:

A precursor, preferably manganese acetate (obtained from Merck, ref. 330825), is mixed with a solution of potassium permanganate (obtained from Merck, ref. 1.09930) in a ratio in mol/l of 1.8/1.2 for at least 24 h at room temperature between 21 and 25° C. Afterwards it is filtered and rinsed with deionized water. The obtained powder is calcined for several hours, preferably 72 h, with an increase in temperature of 6° C./min, preferably until a temperature between 280 and 300° C. is reached. The result is rinsed with deionized water and consecutively dried at 100° C. until the powdered manganese monoxide is obtained which can then be used. In order to obtain a well useable powder, the liquid is preferably fully evaporated.

The low temperature below 300° C. is used to avoid the formation of the more stable but undesired manganese dioxide.

If the adsorbent is a synthetic hydrophilic zeolite of type A, it is preferably synthesized as follows:

Step 1: Providing a sodium hydroxide solution by mixing of sodium hydroxide, for example pellets of sodium hydroxide (obtained from Merck, ref. 567530), in distilled water with a ratio of 110:1 to obtain a homogenous sodium hydroxide solution.

Step 2: Dividing the sodium hydroxide solution into two equal parts, thereby obtaining a first volume and a second volume of sodium hydroxide solution.

Step 3: Crystalline sodium aluminate (obtained from Merck, ref. 13404) is, volume per volume and within 10 to 20 min, mixed to the first volume up to 17% to obtain a solution A.

Step 4: Crystalline sodium silicate Na₂SiO₃ (obtained from Merck, ref. 307815) is, volume per volume, solved in distilled water up to 57% to obtain a solution B.

Step 5: Solution B, is mixed with the second volume, with a ratio of 3.8/1000, to obtain a mixture C, preferably a clear mixture C.

Step 6: The preferably clear mixture C is added to solution A to obtain a mixture D.

Step 7: Mixture D is heated until the water is fully evaporated.

Step 8: Mixture D is cooled by lowering the temperature until a solid E appears.

Step 9: The solid E is filtered and rinsed with distilled water until a pH-value between 8.5 and 9.5 of the rinsing water is obtained.

Step 10: Drying the solid E for a period of 8 h to 15 h, preferably 12 h, at a temperature between 100 and 120° C., preferably 110° C., to obtain the synthetic hydrophilic zeolite of type A.

Step 11: Grinding the synthetic hydrophilic zeolite of type A into a powder.

Preferably, in step 7, the temperature is between 75 and 130° C., preferably 100° C. for 3 h to 4 h. It is the target to fully evaporate the water from mixture D.

Preferably, in step 8, mixture D is cooled by lowering the temperature to a value lower than in step 7 but above room temperature. This cooling allows to avoid the forming crystals of solid E to stick to the bottom of the container in which mixture D is initially located. In one specific embodiment of step 8, the temperature is lowered to 30° C., considering that room temperature is between 20 and 25° C., so that it is easy to remove solid E without it sticking to the bottom.

In a usual manner the pH-value of the rinsing water in step 9 is measured with a pH meter or any other measuring device known by the person skilled in the art that allow a determination of the pH-value. By measuring the pH value of the rinsing water indirectly the surface pH-value of solid E, which will form the synthetic hydrophilic zeolite of type A is measured.

It is preferred that the surface of the zeolite is slightly alkaline, since this enhances the formation of van der Waals forces with the titanium dioxide and the manganese monoxide, which have a slightly acidic surface. This facilitates to generate a homogenous catalytic composition.

In step 10 the drying duration and temperature of solid E allow a gradual removal of the water molecules on the surface of the zeolite. Removing the water too rapidly would lead to the risk of crack formation in the surface of the zeolite. This cracks would weaken the important 3D structure. This is why a drying time between 8 and 15 h with a temperature of 100-120° C. is chosen, to progressively remove the water molecules without risking to weaken the structure of the zeolite.

In step 11 the synthetic hydrophilic zeolite of type A can be grinded using any grinding device.

In a particular embodiment, the adsorbent, especially the zeolite, is grinded to achieve a particle size between 0.5 and 2.5 μm. This particle size increases the affinity for certain microorganisms.

A preferred method for mingling the first catalyst, the second catalyst and the adsorbent is to introduce the first catalyst, the second catalyst and the adsorbent, each in a powdered state and either already pre-mingled or separate, into a liquid to form a slurry. This slurry is then preferably vigorously mixed and the liquid is evaporated to form a dry powdered mixture of the first catalyst, the second catalyst and the adsorbent. This mixture can then be used per se or to form the slurry that is coated onto the carrier.

In a detailed and preferred embodiment, in a liquid preferably consisting of 20% alcohol and 80% deionized water, between 5 and 10% of titanium dioxide, between 2 and 6% of manganese monoxide and between 10 and 20% of the zeolite, each in regard to the total mass of the liquid, are mixed. Consecutively the mixture is heated to evaporate the liquid in order to obtain a powdered catalytic composition which can then be used. It is preferably heated to a temperature of 75° C.-130° C. for a period between 24 h and 120 h, preferably 72 h.

The invention also specifically relates to a slurry, formed from the first catalyst, the second catalyst, the adsorbent and deionized water.

According to another aspect, the invention relates to a catalytic device, obtainable via a manufacturing method as described herein.

According to another aspect, the invention relates to a catalytic composition, containing, in weight percent with regard to its total mass and each in a powdered state, between 27% and 30% of a first catalyst having photocatalytic activity, between 11% and 17% of a second catalyst and between 55% and 59% of an adsorbent.

All embodiments and configurations described herein regarding the catalytic device are also preferred configurations and embodiments of the catalytic composition.

If the second catalyst, as preferred, is a low-temperature catalyst, the catalytic composition can also be called a non-thermal, cold or athermal catalyst, since no high heating is necessary to activate the catalyst. Since the first catalyst, the second catalyst and the adsorbent are each provided in the powdered state, the catalytic composition can also be called a powdery non-thermal or athermal catalyst. In one embodiment, the first catalyst having photocatalytic activity is yet photo-activated.

According to a specific and preferred embodiment of the catalytic composition, the first catalyst is titanium dioxide TiO₂, the second catalyst is manganese monoxide MnO and the adsorbent is a zeolite. The zeolite preferably is a synthetic hydrophilic zeolite of type A.

Additionally, this invention relates to an embodiment of the catalytic composition, in which the amount of the first catalyst is lower than 27% and higher than 30%, the amount of the second catalyst is lower than 11% and higher than 17% and the amount of the adsorbent is lower than 55% and higher than 59%, each in weight percent and with regard to their total mass. All further preferred configurations and embodiments described herein apply to this specific embodiment.

According to another aspect, the invention relates to the use of a catalytic composition as described herein and/or a catalyst device as described herein for depolluting a gas, especially air. The gas preferably contains humidity.

According to another aspect, the invention relates to a gas depolluting apparatus, comprising a catalytic device as described herein and/or a catalytic composition as described herein coated onto a carrier, wherein the catalytic device and/or the catalytic composition is at least partially provided within a designated flow path of the gas to be depolluted.

The gas depolluting apparatus comprises a flow path, which is for example determinate by a pipe or a tube or a housing of the gas depolluting apparatus, especially an inner wall of the housing. The gas to be depolluted enters the gas depolluting apparatus through an entry opening, flows through the apparatus along the flow path and exits the apparatus through an exit opening. Preferably the entry opening is provided near a bottom of the apparatus and the exit opening is provided near a top of the apparatus or vice-versa.

An entry filter may be assigned to the entry opening or may be provided behind the entry opening in the direction of flow and before the catalytic device and/or the catalytic composition in the direction of flow. The catalytic composition and the catalytic device is very effective in depolluting, namely cracking gaseous chemical contaminants and biological contaminants. Particulate contaminants on the other hand can only partially be cracked. Therefore the use of a particulate filter as entry filter is helpful.

Preferably the entry filter is designed to filter particulate contaminants that are larger than biological contaminants such as bacteria, viruses and the like. This is reasonable for two reasons. When the entry filter lets biological contaminants pass through, they do not colonize the filter surface, which would be disadvantageous and possibly dangerous. On the other hand, since the catalytic composition and the catalytic device are very efficient in destroying biological contaminants, it is useful to enable the biological contaminants within the gas to reach them so that they can be destroyed instead of being trapped within the entry filter.

An exit filter may additionally or alternatively be assigned to the exit opening or may be provided before the exit opening in the direction of flow and behind the catalytic device and/or the catalytic composition in the direction of flow. The exit filter serves to trap contaminants that were not destroyed by the catalytic treatment. Using an exit filter instead of an entry filter has the advantage that all biological contaminants are guided to the catalytic device and/or the catalytic composition. Those contaminants surviving the treatment can then be trapped by the exit filter. The exit filter preferably is a HEPA filter.

The gas depolluting apparatus preferably contains one or more treatment stages. Each treatment stage is defined by one or more catalytic device and/or catalytic composition being provided at least partially within the flow path. In the simplest embodiment, the gas depolluting apparatus contains precisely one treatment stage, which is defined by precisely one catalytic device or catalytic composition coated onto a carrier.

Preferably the catalytic devices, catalytic compositions or treatment stages extend over the complete cross section of the flow path, so that no gas can bypass it but must flow through or along it. It is possible to form one treatment stage of more than one catalytic device and/or catalytic composition. In this case, they are provided side by side in the direction of flow but not one behind the other in the direction of flow. When catalytic devices and/or catalytic compositions are provided one behind another, they form separate treatment stages.

In a preferred embodiment the gas depolluting apparatus comprises more than one treatment stage, which may be formed from precisely one catalytic device and/or catalytic composition or from more than one catalytic device and/or catalytic composition being provided side by side per stage.

The gas depolluting apparatus preferably comprises at least one suction unit, or a suction unit is assigned to the apparatus. The suction unit is sucking the gas to be depolluted and provides the gas flow through the gas depolluting apparatus. The suction unit can for example be a fan or blower or one or more fans or blowers. Embodiments of the gas depolluting apparatus can be used and/or are designed for depolluting small volumes per time unit and therefore providing a low gas flow, such as 10 liters per hour or more. In other preferred embodiments the gas depolluting apparatus is designed to provide a gas flow through the apparatus of at least 500 m³/h, more preferably at least 1000 m³/h, further preferably at least 1400 m³/h, especially at least 2000 m³/h. The gas flow is the product of the flow velocity and the flow cross-section. When the flow velocity is kept constant, the gas flow is a function of the flow cross-section and thereby correlating to the catalytic surface. Therefore, especially as long as certain maximum gas velocities are observed, the gas flow can be upscaled simply by increasing the flow cross-section.

A gas flow being too rapid might lead to a decrease of the efficiency of the catalytic destruction of various contaminants, due to a short retention time on the catalytic surface. The gas flow velocity is preferably not exceeding 10 m/s, more preferably not exceeding 7 m/s, especially not exceeding 5 m/s. The gas flow velocity is preferably maintained constant during the depolluting process, preferably via the suction unit. The velocity is especially seen as being constant as long as it only varies around a mean value at ±20% in use.

In order to activate the photocatalyst of the catalytic device and/or the catalytic composition, at least one UV radiation source is also provided. The at least one UV radiation source is configured to irradiate one or more of the treatment stages with UV radiation in order to activate their photocatalysts. It can for example be provided within the side walls of the housing or components limiting the flow path to the sides.

The UV radiation source preferably is a UV emitting lamp, such as LED or a fluorescent tube, especially an array of UV emitting lamps.

In a preferred embodiment, the at least one source of UV radiation is arranged in the designated flow path and configured to irradiate the catalytic composition and/or the catalytic device in order to activate the first catalyst.

In this embodiment, the gas to be depolluted can flow along or through the UV radiation source, which preferably is formed as an array of UV emitting lamps, arranged in a way that gas can flow through the array and thereby along the individual lamps.

In a preferred embodiment one source of UV radiation is arranged in the designated flow path and one catalytic device or catalytic composition is arranged upstream and one catalytic device or catalytic composition is arranged downstream of the source of UV radiation. This design can be called a sandwich design, since the UV radiation source is sandwiched between the catalytic devices and/or the catalytic compositions.

Obviously, the UV radiation source, preferably formed as an array of UV emitting lamps, can be sandwiched between two treatment stages, which can each be formed from a single catalytic composition or catalytic device and/or a plurality of catalytic compositions and/or catalytic devices.

This sandwich design offers the advantage, that both, the UV radiation generated by the UV radiation source and the waste heat from the radiation source inevitably occurring can be used efficiently. The waste heat is used to increase the activity of the second catalyst, provided it is a low-temperature catalyst as preferred. Furthermore the UV radiation can be used efficiently, since on both sides of the UV radiation source a photocatalyst is present.

Explorations of the applicants have shown that with the design of one catalytic device, catalytic composition or treatment stage downstream the UV radiation source and one catalytic device, catalytic composition or treatment stage upstream of the UV radiation source provides efficient depollution. It is possible but preferably not the case that further UV radiation sources besides the sandwiched one are provided. In this specific embodiment, preferably only the sandwiched radiation source is provided.

Therefore, in a preferred and specific embodiment, precisely one catalytic device, catalytic composition or treatment stage is provided upstream and/or precisely one catalytic device, catalytic composition or treatment stage is provided upstream of the UV radiation source. Preferably no further catalysts are provided in the gas depolluting apparatus.

In a preferred embodiment, the UV radiation source is provided as an array of more than one UV emitting lamp, preferably of two to six, especially four UV emitting lamps. The lamps are provided in a unit frame which also provides at least one, preferably two bearings or receptacles for each at least one catalytic device, preferably on both sides of the unit frame. In this embodiment, catalytic devices can be introduced into or applied onto the receptacles or bearings of the unit frame, to form a compact depolluting unit, in which the array is preferably sandwiched by at least two catalytic devices. The receptacles can be supporting surfaces, onto which the catalytic devices are positioned and especially affixed. The receptacles can also be slots, into which catalytic devices, formed as slide-in-modules, can be slid in. In one embodiment of the invention, the depolluting unit is the gas depolluting apparatus.

The gas depolluting apparatus can be used in a manifold of areas. It can for example be used to depollute air in hospitals, preferably in operation theatres or on quarantine stations. Furthermore a small version of the apparatus can for example be used on tables in restaurants or offices for in situ depollution of air being exchanged between persons sitting opposite each other at the table.

As will be described below, the apparatus has shown great efficiency in reducing the amount of a certain coronavirus in the air. It is therefore also a possible and preferred use of the catalytic device, the catalytic composition and/or the gas depolluting apparatus to be used in the treatment of air possibly or definitely containing the coronavirus SARSCoV-2 for reducing or removing the virus in/from the air.

### Experimental Protocol and Results:

The applicants used different catalytic compositions on multiple organic compounds. The following table shows the rate of elimination of the different compounds in mg of a carbon equivalent of each compound per hour [mgC/h], using different catalytic compositions. The use of the carbon equivalent serves to improve the comparability of the numbers and compensates for the fact that the different compounds have a different number of carbon atoms, which leads to a different amount of degradation reactions depending thereupon. Isopropyl alcohol is abbreviated as IPA and butanone as MEK. The relative amount of the components is displayed in weight percent with regard to the total mass of the composition. Titanium dioxide is abbreviated as T, Manganese monoxide as M and the hydrophilic synthetic zeolite of type A as ZA.

| | Composition | | | | |
|---|---|---|---|---|---|
| | | | G | I | |
| | E | D | T 7%, | T 10%, | F |
| | M | M 24%, | M 53%, | M 22%, | T |
| Name | 100% | ZA 76% | ZA 40% | ZA 68% | 100% |
| Toluene | 0 | 0.16 | 0.05 | 0.08 | 0.15 |
| Pentane | 0.06 | 0.22 | 0.18 | 0.18 | 0.4 |
| Cyclopentane | 0.14 | 0.31 | 0.35 | 0.23 | 0.36 |
| Heptane | 0.16 | 0.19 | 0.13 | 0.15 | 0.4 |
| IPA | 0.15 | 0.34 | 0.34 | 0.2 | 0.35 |
| Ethanol | 0.19 | 1.14 | 0.58 | 0.61 | 0.28 |
| MEK | 0.21 | 0.4 | 0.49 | 0.36 | 0.48 |
| | | Composition | | | |
| | | A | H | B | C |
| | | T 65%, | T 50%, | T 50%, | T 28%, |
| | | M 5%, | M 20%, | M 16%, | M 14%, |
| | Name | ZA 30% | ZA 30% | ZA 34% | ZA 58% |
| | Toluene | 0.07 | 0.21 | 0.16 | 0.24 |
| | Pentane | 0.43 | 0.53 | 0.66 | 0.52 |
| | Cyclopentane | 0.4 | 0.57 | 0.61 | 0.55 |
| | Heptane | 0.48 | 0.63 | 0.61 | 0.64 |
| | IPA | 0.38 | 0.44 | 0.53 | 0.57 |
| | Ethanol | 0.28 | 0.48 | 0.54 | 1.07 |
| | MEK | 0.64 | 0.53 | 0.65 | 0.79 |

As can be taken from the table above, the different compositions showed different elimination rates with respect to the different compounds. It can be seen that, each, the sole titanium dioxide (composition F) and the sole manganese monoxide (composition E) were in general less effective than the combinations of two catalysts and an adsorbent. It can also be seen that the compositions H, B and C showed a greater or equal efficiency compared to compositions F and E as well as the combination of manganese monoxide and the zeolite (composition D). This is true with the exception of ethanol, which was eliminated with a higher efficiency at composition D.

Therefore, the combination of the two different catalysts and the adsorbent show a synergistic effect over the sole catalysts or the combination of manganese monoxide with a zeolite.

For the following investigations, composition C was used, since it showed an overall good elimination profile, but especially on the hydrophilic compounds IPA, ethanol and MEK. The efficient elimination of these hydrophilic compounds gives reason for the assumption that such a composition is also efficient on microbial contaminants, since the cell walls of most microbial contaminants are hydrophilic.

In order to demonstrate the efficiency, the following experiments were performed with biological contaminants, using
bacterial spores of Bacillus subtilis in a concentration between 10⁴ and 10⁶ colony-forming units (CFU)/m³ air,
Legionella pneumophila in a concentration between 10⁴ and 10⁶ CFU/m³,
T2-bacteriophages in a concentration between 10³ and 10⁴ plaque-forming units (PFU)/m³

The experiments were performed in an isolator of class A with 0.8 m³ in which an apparatus having the sandwich design as described above was arranged. The carrier was made of aluminum in the honeycomb shape. The UV radiation source was a UV-C emitting lamp of 18 W.

An aerosol generator is used to provide the air flow within the isolator. Each time the air comprises only one of the mentioned contaminants. The contaminated air is then treated with the apparatus.

In order to demonstrate the efficiency of the treatment, a bio collector is used to collect samples from the air before and after the treatment, to provide a comparison. The samples are used to prepare a culture in a medium corresponding to the contaminant used.

For Legionella pneumophila a BYCE medium obtained from Biomerieux is used, containing agar and L-cysteine.

For Bacillus subtilis an LB Luria Bertani medium is used.

For the T2-bacteriophages a medium comprising E. Coli BAM was used and the number of lysis plagues deriving from active virus was examined.

The results show a decrease of 2 log (99.45%) for Legionella pneumophila, 1 log (96.67%) for spores of Bacillus subtilis and 3 log for the bacteriophages T2 (99.98%) after the treatment compared to the air before treatment.

Furthermore additional similar experiments were performed using different biological and chemical contaminants. These were performed within a microbiological safety cabinet having a volume of 0.537 m³. The apparatus was running for 10 minutes. Afterwards and beforehand, the samples were collected for comparison. No outlet filter was used.

The reduction of human coronavirus strain 229E (H-CoV-229E) was >log 2.2 (>99.4%).

The reduction of Staphylococcus aureus CIP 4.83 after a running time of 15 minutes was log 1.3 (94.9%).

Another similar set of tests was performed using different biological contaminants. These were performed with the apparatus having the sandwich design and a HEPA exit filter. The flow rate was 1000 or 1400 m³/h and the duration 6 minutes.

The efficiency for removing Staphylococcus epidermidis (ATCC 14 990) was >99.88% and for removing aspergillus brasiliensis (ATCC 16 404)>99.75% at 1400 m³/h. The efficiency for removing Staphylococcus epidermidis (ATCC 14 990) was >99.91% and for removing aspergillus brasiliensis (ATCC 16 404)>99.82% at 1000 m³/h.

The same tests were performed without the use of the HEPA filter:

The efficiency for removing Staphylococcus epidermidis (ATCC 14 990) was 92.94% and for removing aspergillus brasiliensis (ATCC 16 404) 93.59% at 1400 m³/h. The efficiency for removing Staphylococcus epidermidis (ATCC 14 990) was 96.32% and for removing aspergillus brasiliensis (ATCC 16 404) approx. 90.00% at 1000 m³/h.

Additionally the removal of airborne cat allergens (Fel d 1) was examined using the HEPA filter and a flow rate of 1400 m³/h. The efficiency lay between >99.80% and >99.86%.

Also the reduction of VOC was examined, applying an air flow of 1000 m³/h using filters (A) and without using filters (B) and 1400 m³/h using filters (C) and without the use of filters (D).

The efficiency for removing acetaldehyde was 31.5%±20% (A), 39.4%±11.1% (B), 45.5%±11.0% (C) and 56.2%±8.2% (D).

The efficiency for removing acetone was 98.1%±1.7% (A), 94.5%±0.5% (B), 90.5±1.3% (C) and 100%±2.3% (D).

The efficiency for removing acidic acid was 99.7±0.1% (A), 99.3±0.2% (B), 99.5%±0.10% (C) and 99.4%±0.1% (D).

The efficiency for removing heptane was 98.0±0.2 (A) and toluene 98.4%±0.1% (A).

Embodiments of the invention will be explained with respect to the attached figures.

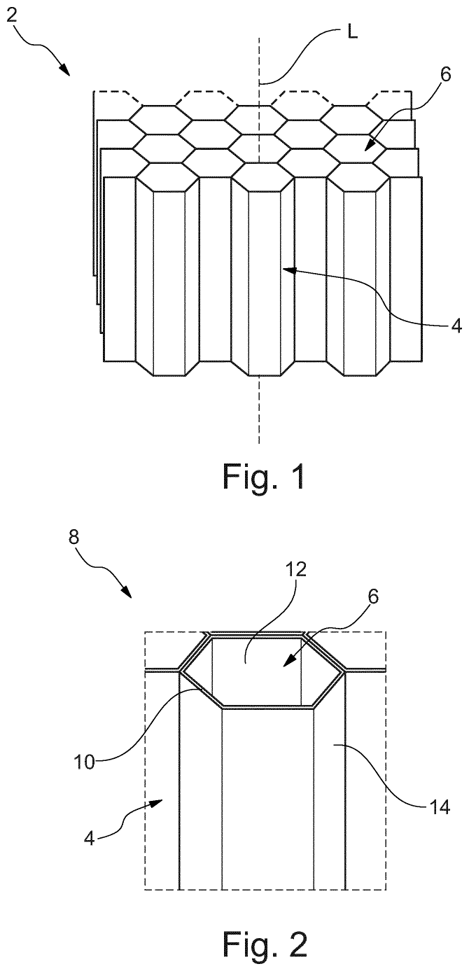

FIG. 1 shows a perspective view onto an embodiment of carrier for a catalytic device,

FIG. 2 shows a perspective section of the carrier of FIG. 1 in a coated state, thereby forming an embodiment of a catalytic device,

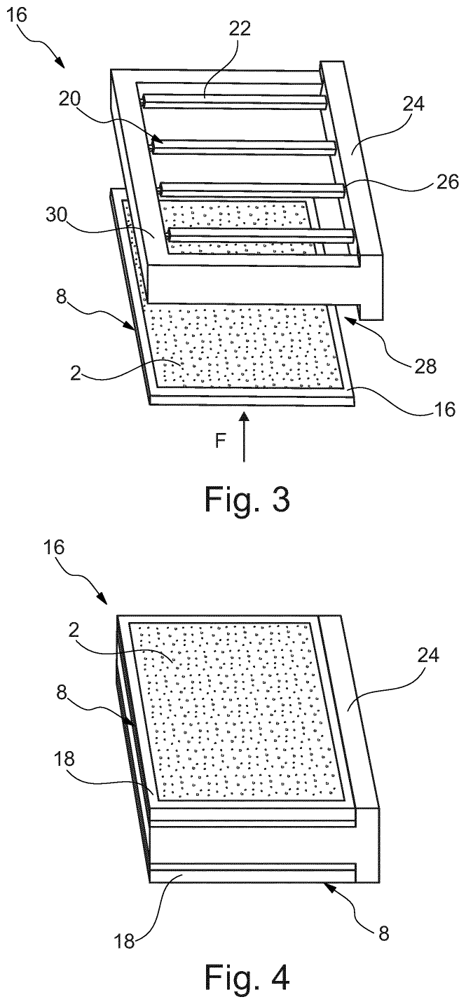

FIG. 3 shows an exploded view of a partially assembled depolluting unit of an embodiment of the gas depolluting apparatus, and

FIG. 4 shows a perspective view of the assembled depolluting unit from FIG. 3.

In FIG. 1, a perspective view onto a solid grid-like carrier 2 is shown. The dotted lines indicate, that only a section of the complete carrier is depicted. The carrier 2 is designed as a honeycomb panel, which preferably is formed from sheets being corrugated to form combs 4. The sheets are preferably made of an inert material such as aluminum. Each comb 4 forms a through hole 6, which allows a gas to flow through the carrier 2.

The carrier 2 has a longitudinal axis L, along which the length of the combs 4 extends. The combs 4 each have a hexagonal cross section, which preferably remains constant along their length. Correspondingly, the through holes 6 also have a hexagonal cross section.

In FIG. 2 a section of a carrier 2 as shown in FIG. 1 is shown in a coated state, thereby forming a catalytic device 8. One comb 4 is depicted with two hinted sidewalls of adjacent combs 4. In the coated state, the surface of the carrier 2 is almost, preferably completely coated with the first catalyst, the second catalyst and the adsorbent.

However, in FIG. 2, the coating 10 is depicted only on the edges of the combs 4 for clarity reasons. Notwithstanding this, the coating 10 is also applied to the inner surfaces 12 of the combs 4 as well as to the outer surfaces 14 of the combs 4, which—aside from the side ends of the carrier 2—form inner surfaces 12 of adjacent combs 4. The surfaces are coated with multiple layers to form a resulting layer having a thickness of preferably 100 μm to 250 μm.

FIG. 3 shows a partially assembled embodiment of a depolluting unit 16 of an embodiment of the gas depolluting apparatus. In one embodiment of the invention, the depolluting unit 16 is the gas depolluting apparatus. In another preferred embodiment, the gas depolluting apparatus comprises a housing, which defines a designated flow path for the gas to be depolluted. The depolluting unit 16 is then arranged within this designated flow path.

The depolluting unit 16 comprises two catalytic devices 8, of which only one is depicted in FIG. 3, with carriers 2 that are coated with the first catalyst, the second catalyst and the adsorbent. The carriers 2 are each formed from honeycomb panels, which are encased by a carrier housing 18 on their side ends. When the carriers 2 are encased in a carrier housing 18, it is possible but not necessarily the case, that only those combs 4 and/or surfaces of the combs of the carrier 2 are coated, which are exposed to the environment and not covered by the carrier housing 18.

The depolluting unit 16 further comprises a UV radiation source 20, designed as an array of four UV radiation emitting lamps 22. Preferably these are lamps emitting UV-C radiation. The lamps 22 are arranged within a unit frame 24. The unit frame 24 preferably comprises sockets 26 for removably mounting the UV emitting lamps 22. Preferably the unit frame 24 furthermore comprises at least one power supply for the UV emitting lamps 22.

The unit frame 24 also comprises two receptacles 28 to accommodate the catalytic devices 8. In FIG. 3 the receptacles 28 are formed by the supporting surfaces 30, to which the carriers 2 and the carrier housings 18 are designed correspondingly. They are either simply positioned to lie on the supporting surfaces 30 or attached to the unit frame 24 via clamps or the like. In another embodiment, the receptacles 28 are designed as slots corresponding to the catalytic devices 8 being designed as slide-in-modules.

The gas to be depolluted flows through the carriers 2 and the UV radiation source 20 along the direction of flow F. Obviously, the direction of flow F, depending on the design of the gas depolluting apparatus, can also run in the opposite direction. The direction of flow F especially runs along the longitudinal axis L.

One advantage of the depolluting unit 16 being designed as depicted is, that the catalytic devices 8 and correspondingly the catalysts within the coating 10 of the carriers 2 are spatially very close to the UV radiation source 20. Therefore, the UV radiation can be used well for activating the photocatalyst, especially because on both sides of the UV radiation source 20 a catalytic device 8 is present. In addition, the waste heat of the lamps 22 can be used to enhance the catalytic activity of the second catalyst, which preferably is a low-temperature catalyst.

FIG. 4 shows the depolluting unit 16 in an assembled state with two catalytic devices 8, attached to the unit frame 24, each abutting the support surfaces 30 of the receptacles 28. It can be seen, that the dimensions of the catalytic devices 8 are corresponding to those of the unit frame 24.

### REFERENCE SIGNS

2 carrier
4 comb
6 through hole
8 catalytic device
10 coating
12 inner surface
14 outer surface
16 depolluting unit
18 carrier housing
20 UV radiation source
22 UV emitting lamp
24 unit frame
26 socket
28 receptacle
30 support surface

1. A method for manufacturing a catalytic device, comprising:
a) providing a first catalyst having photocatalytic activity, a second catalyst which is a different molecule than the first catalyst, and an adsorbent, each in a powdered state,
b) mingling the first catalyst, the second catalyst and the adsorbent to form a catalytic composition and suspending the catalytic composition in a suspension liquid to form a slurry, and
c) repeatedly coating the slurry onto a solid grid-like carrier having a plurality of through holes, wherein the carrier configured to allow a gas to flow through the carrier, and evaporating the suspension liquid.

2. The method according to claim 1, wherein the through holes account for at least 80% of a volume of the carrier.

3. The method according to claim 1 wherein the slurry is binder-free.

4. The method according to claim 1 wherein the slurry is coated onto the carrier via spray-coating.

5. The method according to claim 1 wherein the first catalyst is titanium dioxide.

6. The method according to claim 5, wherein the titanium dioxide is in a form of a mixture of anatase and rutile with an anatase/rutile ratio between 60/40 and 99/1.

7. The method according to claim 1 wherein the second catalyst is a low-temperature catalyst.

8. The method according to claim 1 wherein the adsorbent is a zeolite.

9. The method according to claim 1 wherein the providing step provides in weight percent with regard to their total mass:
between 27% and 30% of the first catalyst, between 11% and 17% of the second catalyst, and between 55% and 59% of the adsorbent.

10. A catalytic device obtained by a method according to claim 1.

11. A catalytic composition, comprising in weight percent with regard to its total mass and each in a powdered state, between 27% and 30% of a first catalyst having photocatalytic activity, between 11% and 17% of a second catalyst which is a different molecule than the first catalyst, and between 55% and 59% of an adsorbent.

12. The catalytic composition according to claim 11, wherein the first catalyst is titanium dioxide the second catalyst is manganese monoxide and the adsorbent is a zeolite.

13. The catalytic composition according to claim 11, wherein the adsorbent is a synthetic hydrophilic zeolite of type A.

14. The catalytic composition according to claim 11, wherein the first catalyst is photo-activated.

15. The catalytic composition according to claim 11, being a non-thermal catalyst, and comprising, in weight percent with regard to its total mass:
between 27% and 30% of photo-activated titanium dioxide as the first catalyst,
between 11% and 17% of manganese monoxide as the second catalyst,
between 55% and 59% of synthetic hydrophilic zeolite of type A as the adsorbent.

16. A gas depolluting apparatus, comprising a catalytic device according to claim 10 and/or a catalytic composition comprising in weight percent with regard to total mass and each in a powdered state, between 27% and 30% of a first catalyst having photocatalytic activity, between 11% and 17% of a second catalyst which is a different molecule than the first catalyst, and between 55% and 59% of an adsorbent coated onto a carrier, wherein the catalytic device and/or the catalytic composition is at least partially provided within a designated flow path of gas to be depolluted.

17. The gas depolluting apparatus according to claim 16, further comprising at least one source of UV radiation arranged in the designated flow path and configured to irradiate the catalytic composition and/or the catalytic device in order to activate the first catalyst.

18. The gas depolluting apparatus according to claim 17, wherein the at least one source of UV radiation is arranged in the designated flow path and the catalytic device or catalytic composition is arranged upstream and a second catalytic device identical to the catalytic device or a second catalytic composition identical to the catalytic composition is arranged downstream of the at least one source of UV radiation.
